(19) United States
(12) Patent Application Publication
(10) Pub. No.: US 2010/0318031 A1
(43) Pub. Date: Dec. 16, 2010

(54) External Stylette Needle and Method of Use

(76) Inventor: Kevin Henry, Peoria, IL (US)

Correspondence Address: Robert L. Knechtel, 605 S. Valley Hill Road, Bull Valley, IL, 60098, US

(21) Appl. No.: 12/482,833

(22) Filed: Jun. 11, 2009

(51) Int. Cl. A61B 17/34

(52) U.S. Cl. 604/125; 604/158

(57) ABSTRACT

An external stylette needle device for use in medical/surgical procedures to reduce the introduction of air into a patient and method of use comprising a stylette sizably fit over at least a portion of the exterior surface of a hollow core needle shaft such that the stylette can slide about the needle shaft. The needle having a proximal end including a hub and a distal end including a tip and a distal orifice, the needle further comprising a proximal orifice adjacent the hub. The stylette being further adapted to alternately and reversibly close or open the needle distal orifice and the needle proximal orifice. The device may include an electromagnetic charge to ensure that the stylette remains in contact with the external surface of the needle.

### I. TECHNICAL FIELD

The present invention relates to needles used in medical procedures, and, more particularly, to an external stylette needle device comprising a needle having an stylette sizable fit to the external length of the needle shaft and, further, being adapted to slide about the needle shaft to alternately and reversibly close or open needle orifices.

### II. BACKGROUND OF THE INVENTION

One undesired result of epidural procedures is paraplegia. Historically, attempts to minimize this and other risks have generally focused on needle configuration. The major concentration in this regard has been on the use of blunt tip needles. However, a shortcoming of these needles is that they can cause excessive tissue trauma and, further, enable the introduction of non-particulate steroid which is thought to cause end artery, or arteriole, infarction of spinal cord tissue.

Another proposed mechanism for post-procedure paraplegia is the introduction of air into a radicular artery during the procedure. The theory is that an air embolus blocks distal blood flow resulting in spinal cord injury. One technique to minimize this event involves filling the needle with fluid and tapping the shaft of the needle to break up surface tension of the fluid, which in turn, enables the user to evacuate accumulated air. It is recognized that this technique does not remove all air from a needle, potentially resulting in enough air left in the needle shaft that injury can result.

Another conventional technique to minimize retained air is the use of a stylette in conjunction with a needle. Currently, stylettes consist of a thin shaft of solid material that is passed into the internal core of the needle. Following the introduction of the needle, the stylette is then withdrawn so that injection can occur. Once injection is complete and the user desires to withdraw the needle, the stylette can be reintroduced, if need be. The stylette functions to obliterate the needle tip, thereby, in theory, reducing the opportunity for air to accumulate. A shortcoming of the conventional internal stylette is that a column of air fills the cavity created by its removal. Another shortcoming is that removal of the stylette and its reinsertion, if needed, require time, thereby unnecessarily lengthening procedure time. Yet another shortcoming of the conventional stylette is that its reinsertion increases the chance of infection.

Accordingly, there is need for a device that increases the margin of safety of procedures by reducing the risk of air introduction to sub-clinical levels, reducing procedure time and, further, reducing attendant infection and non-particulate matter introduction risks. The present invention provides such a device.

### III. OBJECTS AND ADVANTAGES OF THE PRESENT INVENTION

It is an object of the present invention to provide an externally positioned stylette for use with a needle.

It is another object of the invention to provide a nerve block needle adapted to accept an external stylette.

It is yet a further object of the invention to provide an external stylette needle device that enables the evacuation of air from the needle.

One advantage of the inventive device is it reduces to subclinical levels the opportunity for air introduction into a patient during a procedure.

It is a further advantage of the invention that the time for performing a procedure is reduced by eliminating the need for stylette removal and reinsertion.

It is yet a further advantage of the invention that the risk of infection is reduced by eliminating the need to reintroduce a stylette during a procedure.

It is yet a further advantage of the invention that the risk of non-particulate matter introduction is reduced.

The present invention provides for the above objects and advantages, and others as will be understood by those skilled in the art, through an external stylette needle device comprising a stylette sized to fit slidably about the exterior surface of a needle to alternately and reversibly obliterate one or more needle orifices as desired by the user.

### IV. SUMMARY OF THE INVENTION

The above objects and advantages are provided for in the present inventive external stylette needle device. The device comprises a stylette sizably fit over at least a portion of the exterior surface of a needle shaft such that the stylette can slide about the shaft, the stylette further having a proximal end and a distal end. The needle having the well known configuration comprising an elongated hollow structure having a proximal end further including a hub for connecting to a delivery vehicle, such as a syringe, and a distal end including a tip for introduction of the needle to a target site and a distal orifice. The needle being adapted to further include a proximal orifice adjacent the hub. The stylette further being adapted to alternately and reversibly close or open the needle distal orifice and the needle proximal orifice.

In use, the stylette is fitted over the exterior surface of the needle shaft. Once the needle is inserted to the desired location, the stylette is positioned such that the stylette distal end is clipped into the needle distal orifice, thereby closing the needle distal end. Fluid, for example a medicant or dye, is then introduced into the needle through the hub by asserting pressure which, in turn, forces the fluid and air within the needle to move up through the hollow needle shaft and expel through the now open needle proximal orifice. Once the fluid expels without sign of containing air, the stylette is slid along the exterior surface of the needle such that the needle distal orifice opens and the proximal needle orifice is closed by the proximal end of the stylette. If required, the stylette can be variously moved about the exterior surface of the needle as is required by the procedure or the desire of the user.

In an alternate embodiment, the device may include an electromagnetic charge to ensure that the stylette remains in contact with the external surface of the needle.

There has been outlined, rather broadly, the more important features of the invention in order that the detailed description thereof that follows may be better understood, and in order that the present contribution to the art may be better appreciated. There are, of course, additional features of the invention that will be described hereinafter and that will form the subject matter of the invention.

### V. BRIEF DESCRIPTION OF THE DRAWINGS

FIG. 1 illustrates an exploded side view of the inventive device depicting the stylette separate from the needle.

FIG. 2 illustrates a top cut away view below the hub of external stylette depicting its curved configuration adjacent the needle.

FIG. 3 illustrates a side view of the inventive device wherein the stylette is affixed to the needle.

FIG. 4 illustrates a side view of the inventive device wherein the stylette includes additional, but non-limiting, attachment members.

FIG. 5 illustrates a top view below the hub of the needle and the lever of the stylette depicting an alternate embodiment of stylette wherein its entire length is of a size that it serves as an attachment member to secure the stylette to the needle.

FIG. 6 illustrates a top cut away view below the hub and the lever of the stylette of the inventive device depicting a representation of an electromagnetic charge where the electromagnetic charge is illustrated schematically in a box with a magnified perspective.

FIG. 7A illustrates a front view of the distal end of a needle depicting a representative distal orifice configuration.

FIG. 7B illustrates a front view of the distal end of an alternative needle distal orifice configuration.

FIG. 7C illustrates a front view of the distal end of a second alternative needle distal orifice configuration.

FIG. 7D illustrates a side view of the distal end of a needle having a curved tip.

FIG. 8 illustrates a front view of the inventive device in which the stylette is in a closed distal orifice-open proximal orifice position.

FIG. 9 depicts a cut away top view at the level of the distal needle orifice of the inventive device illustrating the distal end of the stylette clipped into the distal orifice of the needle.

FIG. 10 illustrates a front view of the inventive device in which the stylette is in an open distal orifice-closed proximal orifice position.

FIG. 11 illustrates the inventive device in use wherein the stylette is in a closed distal orifice-open proximal orifice position with a fluid containing syringe affixed to the needle hub.

### VI. DETAILED DESCRIPTION OF THE PREFERRED EMBODIMENT

Before explaining the preferred embodiment of the present invention in detail, it is to be understood that the present invention is not limited in its application to the details of arrangements of the components set forth in the following description. The present invention is capable of other embodiments and of being practiced and carried out in various ways. Also, it is to be understood that the phraseology and terminology employed herein are for the purpose of description and should not be regarded as limiting. It is also to be understood that where ranges are provided for various aspects of the invention and for examples, they are approximate ranges and are not to be limiting except where noted otherwise.

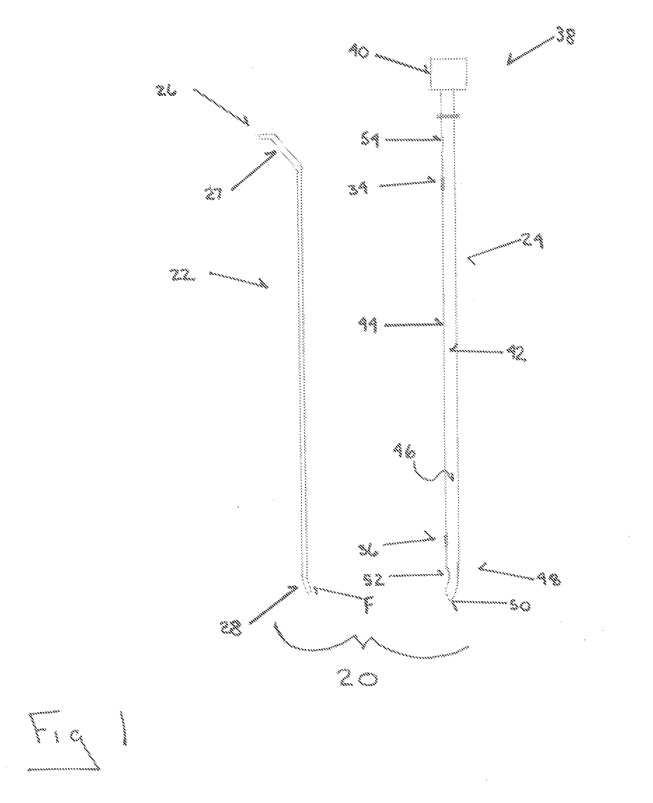

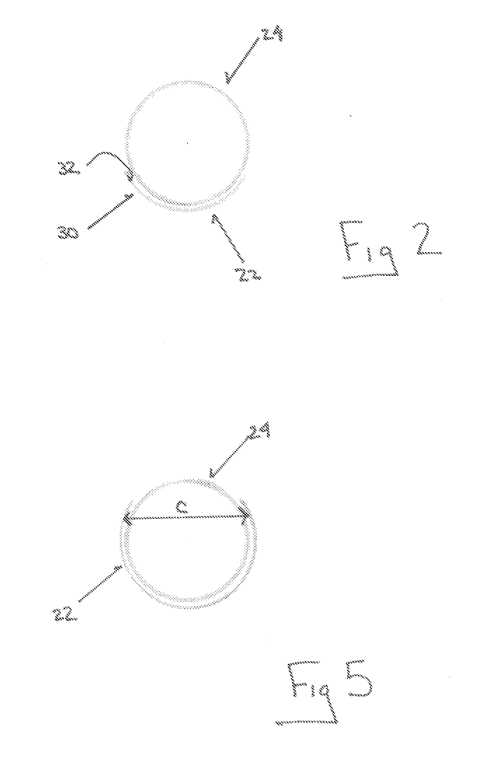

Turning to FIG. 1, an exploded view of the inventive device 20 is depicted wherein the external stylette 22 is separated from the needle 24. As can be seen, stylette 22 is an elongated structure having a proximal end 26 further including lever 27 and a distal end 28. As seen in FIG. 2, stylette 22 is contoured having a convex outer surface 30 and a concave inner surface 32 along its length. While depicted in such a manner as current needles have generally circular shafts, those skilled in the arts will quickly understand that the shape is not critical Returning to FIG. 1, needle 24 further includes proximal attachment member 34 and distal attachment member 36. Needle 24 has the general configuration of a standard hollow core needle including a first end 38 including a hub 40, a shaft 42 having an exterior surface 44 and an interior hollow core 46, and a second end 48 including a tip 50 and a distal orifice 52. As can be seen, needle 24 further comprises proximal orifice 54 adjacent hub 40 and first end 38.

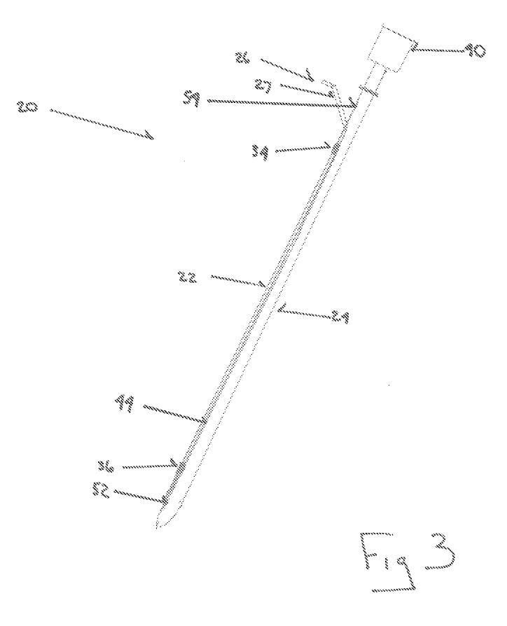

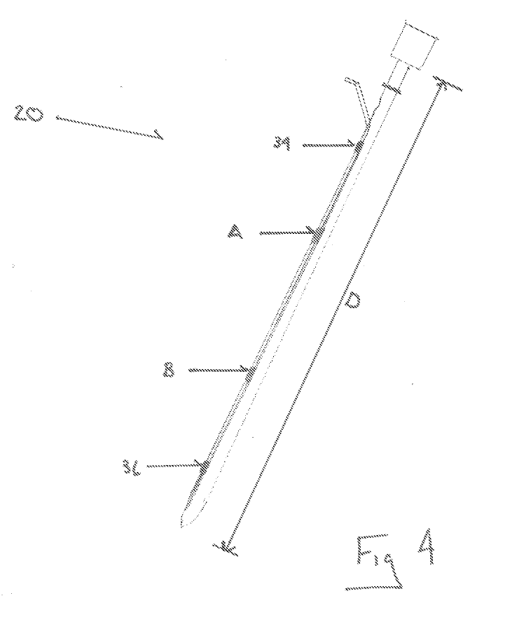

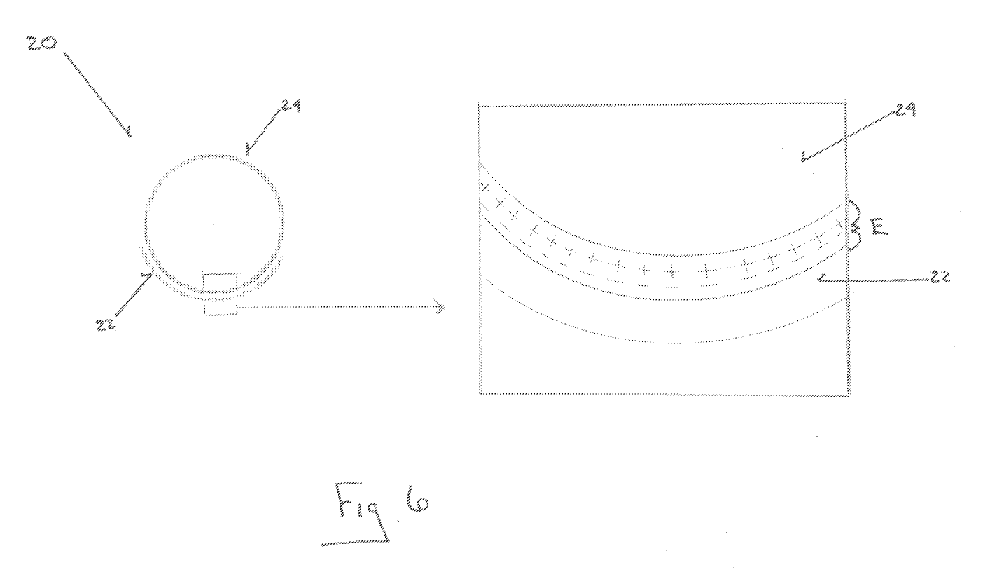

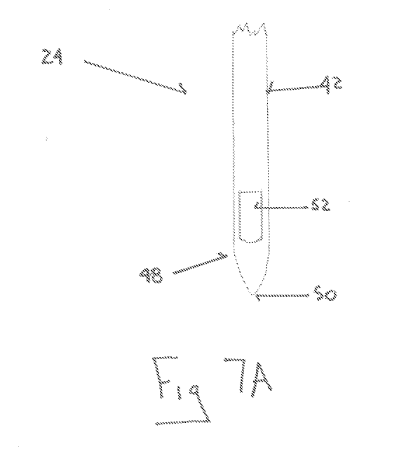

Turning to FIG. 3, it is seen that stylette 22 is movably affixed to needle 24 by means of stylette proximal attachment member 34 and distal attachment member 36. As is further seen, Stylette 22 fits snuggly against exterior surface 44 and is of a length such that it alternately fits over the entirety of either needle proximal orifice 54 or distal orifice 52. Stylette proximal attachment member 34 and distal attachment member 36 should provide sufficient securing force that stylette 22 maintains close proximity to needle shaft 42 along its entire length yet enable stylette 22 to move freely along the length of shaft 42. Accordingly, depending on the particular application or required length for inventive device 20, additional attachment means beyond proximal attachment member 34 and distal attachment member 36 may required. If needed, additional attachment members may be utilized (as seen in FIG. 4 and depicted by non-limiting attachment members A and B). Moreover, and as depicted in FIG. 5, stylette 22 may be sized C such that its entire length D (FIG. 4) is of a size and configuration that it serves as an attachment member, or, alternatively, and as depicted in FIG. 6, an electromagnetic charge E may be applied to the device to ensure stylette 22 maintains contact with needle 24. Additional means and methods may also be employed.

Stylette 22 is preferably made of a material that can be sterilized, has suitable tensile and compression strength properties so that it does not buckle, bend, or break during introduction and of a thickness sufficient to be effective but does not add appreciable bulk to the diameter of needle 24. According, surgical steel is preferred as the material of choice for the stylette 22 of inventive device 20.

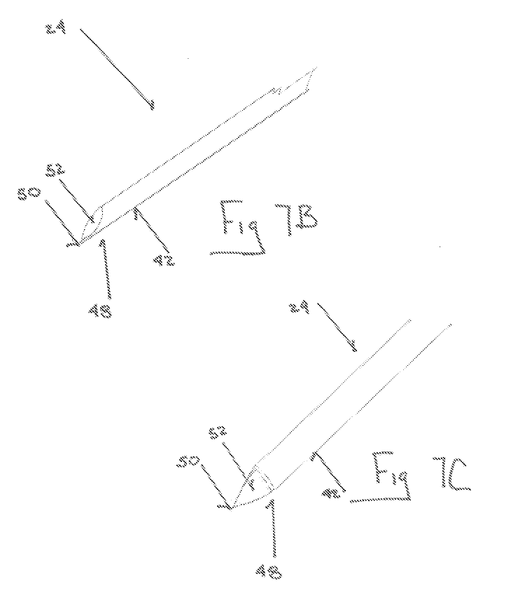

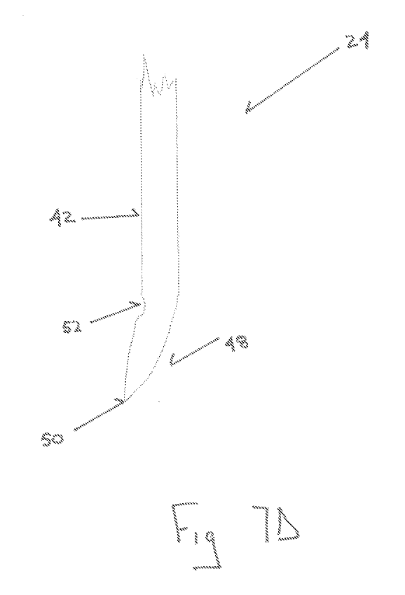

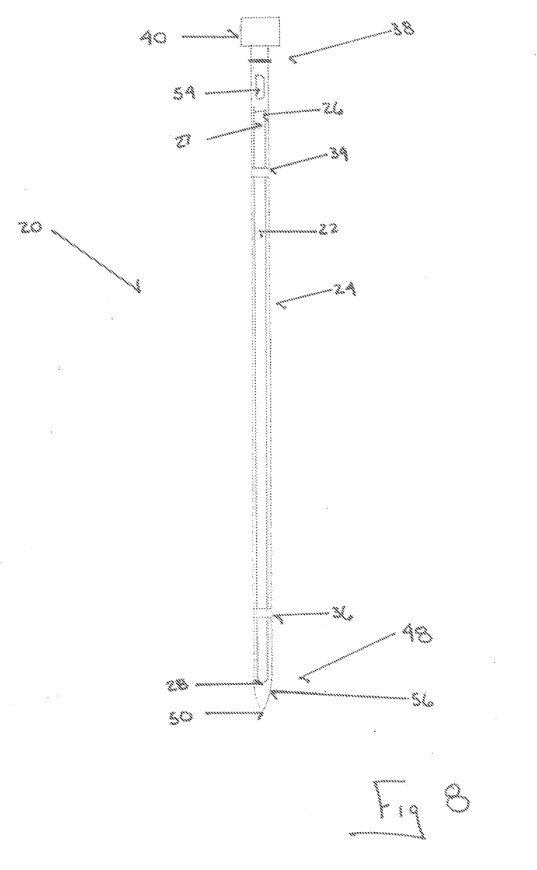

As is well known in the arts, there are many needle configurations and variations. Each user has his or her requirements as dictated by the intended procedure and the personal choice of the user. For example, needle tips and distal orifices may be configured in what are commonly known as the Whitaker style (FIG. 7A), Touy style (FIG. 7B) or Quinke style (FIG. 7C). Additionally, the needle tip may be curved (FIG. 7D). As will be appreciated by those skilled in the arts, the inventive device is suitable for these and other configurations. Only the length of stylette 22 need be varied to ensure that it can simultaneously and alternately closed and open proximal orifice 54 and distal orifice 52.

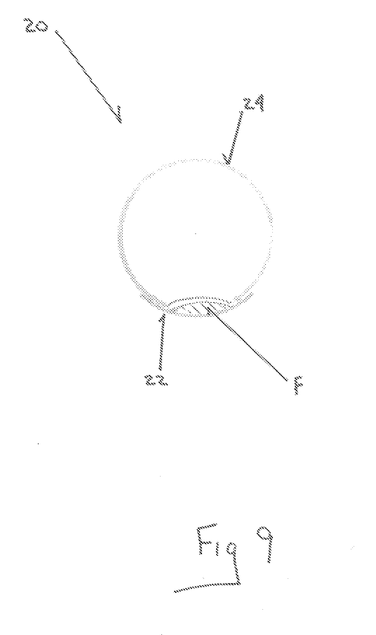

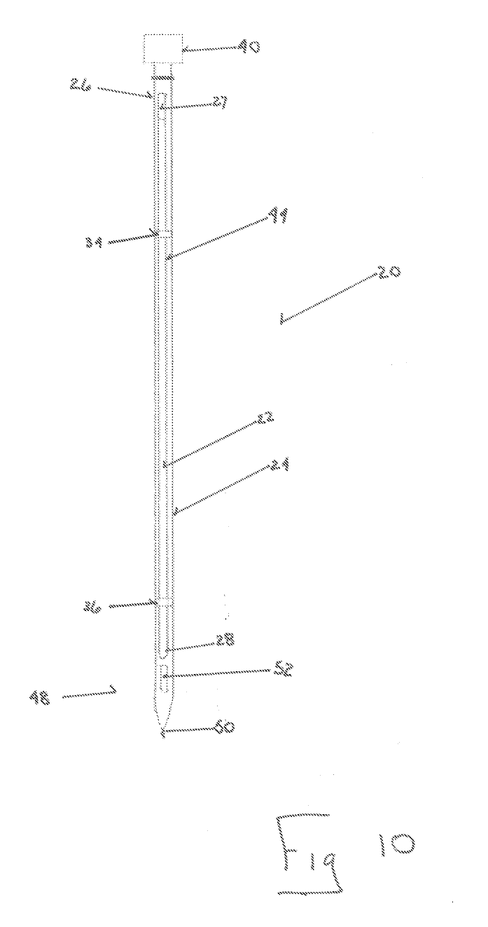

Turning to FIG. 8, inventive device 20 is depicted with stylette 22 in a distal orifice closed-proximal orifice open position with respect to needle 24. In this position, stylette distal end 28 is clipped into distal orifice 52 such that stylette distal end 28 completely covers distal orifice 52. As tip 50 of needle 24 is generally tapered 56, stylette distal end 28 must be sufficiently curved in an inward direction F (FIG. 1 and FIG. 9) such that as it is positioned over distal orifice 52, it moves to conform to the taper of tip 50 and cover the entirety of distal orifice 52. When distal orifice 52 is covered by distal end 28, proximal orifice 54 will be in an open position.

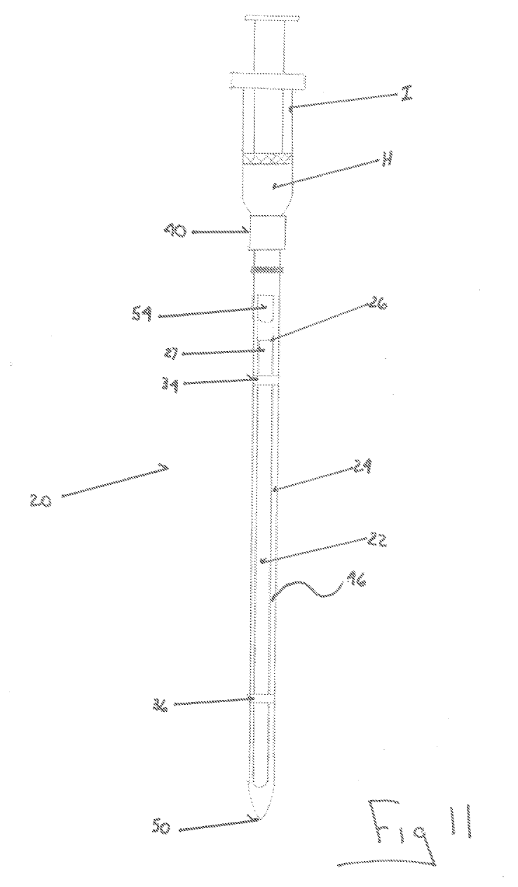

Conversely, and as depicted in FIG. 10, when stylette 22 is manipulated to open distal orifice 52 by sliding it along the length of shaft 42, proximal orifice 54 will close.

In use, inventive device 20 is positioned into target site (not shown for clarity). Turning to FIG. 11, once positioned and stylette distal end 28 is in a distal orifice closed position, fluid H is introduced, for example by a syringe I, into needle interior hollow core 46 through needle hub 40. This, in turn, forces the fluid and any air contained within hollow core 46 to move up through the hollow core 46 and expel through open needle proximal orifice 54 until the air is evacuated and the fluid expels without sign of containing air through proximal needle orifice 54. The user then slides stylette 22 along exterior surface 44 employing lever 27 until needle distal orifice 52 opens which is signaled by the closure of proximal needle orifice 54 (FIG. 10). Once in the open distal orifice-closed proximal orifice position, the user may complete the procedure injection.

If needed, stylette 22 can be variously moved about exterior surface 44 of needle 24 as is required by the procedure or the desire of the user. For instance, the user may desire that for withdrawing of needle 24 that stylette 22 be in a closed distal orifice-open proximal orifice position.

It is to be understood, however, that even though numerous characteristics and advantages of the preferred and alternative embodiments have been set forth in the foregoing description, together with details of the structure and function of the embodiments, the disclosure is illustrative only, and changes may be made in detail within the principles of the invention to the full extent indicated by the broad general meaning of the terms in which the appended claims are expressed.

I claim:

1. A Stylette for use with a needle having an external surface, the Stylette being adapted to movably affix to the external surface of the needle.

2. The Stylette of claim 1 further being a predetermined length and having an outer surface and an inner surface, the inner surface contoured in shape to compliment the external surface of the needle.

3. The Stylette of claim 1 further comprising a convex outer surface and a concave inner surface along the length of the stylette.

4. The Stylette of claim 1 further comprising at least one attachment member for affixing to the needle.

5. The Stylette of claim 1 further comprising an electromagnetic field for affixing the stylette to the needle.

6. The Stylette of claim 1 further having a proximal end and a distal end, the distal end being inwardly curved.

7. A stylette needle device comprising a needle having an external surface and a stylette movably affixed to the external surface of the needle.

8. The Device of claim 7 wherein the stylette further being a predetermined length and having an outer surface and an inner surface, the inner surface contoured in shape to compliment the external surface of the needle.

9. The stylette of claim 8 further comprising a convex outer surface and a concave inner surface along the length of the stylette.

10. The Device of claim 7 wherein the needle further comprises a first end, a second end, and a proximal orifice adjacent to the first end.

11. The Device of claim 7 wherein the stylette further comprises at least one attachment member for affixing to the needle.

12. The Device of claim 7 further comprising an electromagnetic field between the needle and the stylette for affixing the stylette to the needle.

13. The Device of claim 7 wherein the stylette has a proximal end and a distal end, the distal end being inwardly curved.

14. An external stylette needle device comprising a needle having an external surface, an inner hollow core, a first end including a proximal orifice and a second end having a tip and a distal orifice; and,
a stylette movably affixed to the external surface of the needle, the stylette being of a length wherein it alternately and reversibly covers the proximate orifice and the distal orifice.

15. The Device of claim 14 wherein the stylette further having an outer surface and an inner surface, the inner surface contoured in shape to compliment the external surface of the needle.

16. The stylette of claim 15 further comprising a convex outer surface and a concave inner surface along the length of the stylette.

17. The Device of claim 14 wherein the stylette further comprises at least one attachment member for affixing to the needle.

18. The Device of claim 14 further comprising an electromagnetic field between the needle and the stylette for affixing the stylette to the needle.

19. The Device of claim 14 wherein the stylette has a proximal end and a distal end, the distal end being inwardly curved.

20. A stylette for affixing to the external surface of a hollow core shaft having a length, a first end and a second end comprising a proximal end, a distal end, and at least one attachment member, the stylette being further adapted to move along the length of the shaft.

21. A method for performing medical and surgical procedures involving the use of a hollow core needle having an external stylette comprising the steps of:
inserting a needle having a proximal end including a hub and a proximal orifice, a distal end having a tip and a distal orifice and a stylette having a first end, a second end movably affixed to the external surface of the needle into a target site on a patient's body;
manipulating the stylette to a position in which the distal orifice is closed and the proximal orifice is open;
causing a fluid to flow through the needle and expelling through the proximal orifice until such time as any air located in the needle has been evacuated from the needle; and,
manipulating the stylette to a position in which the distal orifice is open and the proximal orifice is closed.

22. The Method of claim 21 including the further step of manipulating the stylette prior to or during the withdrawal of the needle so that the stylette is positioned in such a way that the distal orifice is closed and the proximal orifice is open.

23. A method for performing medical and surgical procedures involving the use of a hollow core needle having an external stylette comprising the steps of:
manipulating a needle having a proximal end including a hub and a proximal orifice, a distal end having a tip and a distal orifice and a stylette having a first end and a second end movably affixed to the external surface of the needle until the stylette is in a position in which the distal orifice is closed and the proximal orifice is open;
inserting the needle into a target site on a patient's body;
causing a fluid to flow through the needle and expelling through the proximal orifice until such time as any air located in the needle has been evacuated from the needle; and,
manipulating the stylette to a position in which the distal orifice is open and the proximal orifice is closed.

24. The Method of claim 23 including the further step of manipulating the stylette prior to or during the withdrawal of the needle so that the stylette is positioned in such a way that the distal orifice is closed and the proximal orifice is open.
